(19) United States
(12) Patent Application Publication
(10) Pub. No.: US 2020/0087703 A1
(43) Pub. Date: Mar. 19, 2020

(54) METABOLIC LABELING OF BACTERIAL TEICHOIC ACIDS CELL WALL

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d'Hères (FR)

(72) Inventors: Thierry Vernet, Grenoble (FR); Yung-Sing Wong, Saint-Martin-d'Hères (FR); Anne-Marie Di Guilmi, Saint-Paul-de-Varces (FR)

(73) Assignees: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d'Hères (FR)

(21) Appl. No.: 16/615,012

(22) PCT Filed: May 22, 2018
(86) PCT No.: PCT/EP2018/063337

§ 371 Date: Nov. 19, 2019

(30) Foreign Application Priority Data
May 22, 2017 (EP) 17305596.3

(51) Int. Cl. C12Q 1/16; G01N 33/58

(52) U.S. Cl. CPC C12Q 1/16; G01N 33/582

(57) ABSTRACT

The disclosure provides a new method for the specific metabolic labeling of bacterial teichoic acids cell wall by modified choline and click chemistry, and its use in various applications such as bio-imaging, diagnostic, vaccination or bio-materials engineering.

The invention pertains to the field of bacterial labeling. The present invention provides a new method for the specific metabolic labeling of bacterial teichoic acids cell wall by modified choline and click chemistry, and its use in various applications such as bio-imaging, diagnostic, vaccination or bio-materials engineering.

The bacterial cell wall is composed by peptidoglycan (PG), i.e. a matrix of linear glycan chains of N-acetylmuramic acid and N-acetylglucosamine residues cross-linked via peptides strands made of L- and D-amino acids. Labeling of the cell wall of bacteria is a very challenging task because this cell component displays essential functions (such as mechanical resistance, shape or attachment of other molecules) and, in particular, its assembly is the main Achilles' heel of bacteria targeted by beta-lactams, which encompass over 60% of the total antibiotics used today.

Given that the glycan polymers of the cell wall are not genetically encoded, they cannot be labelled by classic recombinant DNA techniques. Currently, immunolabeling is the main technique used to detect and localize cell surface components. This technique is convenient because it allows the labeling of both genetically and non-genetically encoded molecules but requires specific antibodies against each target molecule. The major disadvantage of this technique is that the cells have to be chemically fixed and permeabilized, a procedure that alters to a great extend the cell surface structure and prevents all kind of live cell imaging, a mandatory condition to decipher biological functions of molecules in a physiological environment.

In this context, a new field of research is emerging which consists in implementing chemical tools in cellular biology. These techniques have been developed for modifying cell surfaces with non-native synthetic compounds. The basic strategy to display small molecules of interest onto the surface of bacteria relies on their metabolic incorporation. These chemically modified molecules are added exogenously in the bacterial culture, taken up by the appropriate machinery and metabolically incorporated into the growing cell wall. Some of the polymers of the bacterial cell wall, such as peptidoglycan (Sadamoto et al., J. Am. Chem. Soc., 124:9018-9019, 2002), lipopolysaccharide (Liu et al., Proc. Natl. Acad. Sci. USA, 106:4207, 2009) or glycolipids (Backus et al., Nat. Chem. Biol., 7:228, 2011), have already been targeted by metabolic labeling.

In the case of Gram-positive bacteria, the cell wall also contains Teichoic Acids (TAs). These glycopolymers are either attached to the peptidoglycan (wall teichoic acids, WTA) or anchored to the cytoplasmic membrane (lipoteichoic acid, LTA). TAs are complex polysaccharides made of a succession of 4-8 repeating units (as illustrated in FIG. 1). In pneumococci, one repeating unit contains AATGalp, Glcp, Rib-ol-5-P, and two GalpNAc, both substituted in position O-6 with Phospho-Choline (P-Cho). All repeating units are α-1-linked, only the first is β-1-linked to the cell anchor. The hydroxyl groups of Ribol-5-P can be substituted in non-stoichiometric amounts by D-Ala. In LTAs, the cell anchor is a Glcp-diacylglycerol. In WTAs, the chain is attached to the MurNAc of the PG by way of a Grop-ManNAc-GIcNAcp linkage unit.

TAs play important roles in host infection and participate to the regulation of cell morphology. In particular, it has been shown that bacteria depleted in TAs display shape defects and irregular wall thickness, indicating an intimate interplay between PG and TAs (Kawai et al., EMBO J., 30:4931, 2001; Santa Maria et al., Proc. Natl. Acad. Sci. USA, 111:12510, 2014). However, despite the essential roles of TA in physiopathological processes, knowledge on TA biosynthesis is hampered by the lack of appropriate methods to trace TA in live cells. Therefore, there is a need for new tools allowing the specific labeling and tracking of TA on live bacteria.

Here, the Inventors provide a new method for the specific labeling of pneumococcal TA by modified choline and click chemistry. Since this method is rapid, cheap and easy-to-use, it can be used for numerous applications, in particular for bio-imaging, diagnostic, vaccination and bio-materials engineering.

This invention results from the unexpected observation made by the Inventors that bacteria can metabolize a modified choline and incorporate it in the teichoic acid of the cell wall, allowing a bioorthogonal labeling reaction with a large variety of tag molecules.

### Method of Labeling

In an aspect, the invention relates to a method of labeling a bacterium that is able to metabolize choline, said method comprising a step (i) of incubating the bacterium in a culture medium containing a modified choline which is metabolized by the bacterium, covalently associated to the TA into the cytoplasm, before being exported and integrated into the cell wall of the bacterium.

Unless defined otherwise, all technical and scientific terms used herein have the same meaning as commonly understood to one skilled in the art. For convenience, the meaning of certain terms and expressions employed in the specification, examples, and claims are provided.

In the invention, the expression “a bacterium that is able to metabolize choline” refers to a group of bacteria that possess the cell machinery required to import the choline present in the medium, to metabolize and to load the choline with teichoic acids.

In particular, the bacterium is a Gram-positive bacterium, which can be selected from the Streptococcus genus, or a Gram-negative bacterium, which can be selected from Haemophilus or Neisseria genera, and in particular from S. pneumoniae, H. influenzae and Neisseria ssp.

Indeed, it is known that the growth of S. pneumoniae is strictly dependent from choline and the phosphocholine (PCho), a derivative of choline, is used for the decoration of the teichoic acids. H. influenzae has PCho containing lipopolysaccharides, whereas Neisseria ssp. have PCho decorated lipopolysaccharides and proteinaceous pili. Both H. influenzae and some Neisseria species share with S. pneumoniae the same transporter (LicB) to import choline from the medium and they both expose PCho groups at their surface (Fan et al., Mol. Microbiol., 50:537, 2003; Serino et al., Mol. Microbiol., 43:437, 2002).

A bacterium able to metabolize choline can also be identified by screening the presence of specific enzymes, such as those encoded by the lic loci (licA, licB, licC and licD1, licD2), as for example the transporter encoded by the gene licB (Accession Number NP_358739.1), its homologous or orthologous genes in S. pneumoniae.

In an embodiment, the invention relates to the method as defined above, wherein the bacterium is selected from the Streptococcus genus, in particular from the species S. pneumoniae and S. mitis, more particularly from S. pneumoniae. In the invention, the choline dependency of pneumococcal growth is harnessed to enable the metabolic labeling with a modified choline.

In an embodiment, the invention relates to the method of labeling a bacterium as defined above, wherein the bacterium is selected from the Haemophilus genus, in particular from the H. influenzae species.

In an embodiment, the invention relates to the method of labeling a bacterium as defined above, wherein the bacterium is selected from the Neisseria genus, in particular from the Neisseria ssp., more particularly from N. lactamica species.

In the invention, the expression “modified choline” refers to a choline that has been modified to integrate a chemical modification allowing the direct detection of the modified choline, i.e. via a direct labeling, or a chemical modification allowing a bioorthogonal reaction of the modified choline with a tag molecule, i.e. via an indirect labeling.

In the case of a direct labeling, the modified choline is chemically modified to incorporate a radioactive isotope.

In an embodiment, the invention relates to the method of labeling a bacterium as defined above, wherein the modified choline comprises a radioactive isotope, such as ³H or ¹⁵N.

In the case of an indirect labeling, the detection of the modified choline requires the addition of a tag molecule via a bioorthogonal reaction.

The expression “bioorthogonal reaction” is a generic and well-known expression that refers to a chemical reaction that is achieved inside or at the surface of a living cell without interfering with native biochemical processes.

In an embodiment, the invention relates to the method of labeling a bacterium as defined above, that further comprises a step (ii) of contacting the bacterium with a tag molecule to generate a binding reaction between the modified choline bound to the TA present in the cell wall of the bacterium and the tag molecule.

In a preferred embodiment, the invention relates to the method of labeling a bacterium as defined above, wherein, in step (ii), the binding reaction between the modified choline bound to the TA present in the cell wall of the bacterium and the tag molecule is made by a click chemistry reaction.

The term “click chemistry” is a generic term that encompasses a wide variety of chemical reactions between pairs of functional groups (or “clickable” reagents) that rapidly and selectively react with each other in aqueous conditions to form a stable conjugate. The click chemistry offers convenient, versatile and reliable two-steps coupling procedures of two molecules (e.g. “A” and “B”) that are widely used in chemical biology, especially in the field of cell labeling, to generate bioorthogonal ligation reactions (for review, King et Wagner, Bioconjugate Chem., 25: 825, 2014).

The cell labeling requires reaction procedures that can be performed under physiological conditions (neutral pH, aqueous solution, ambient temperature) with low reactant concentrations to ensure non-toxic and low background labeling at reasonable time scales while still preserving the biological functions.

Click chemistry reactions is mainly categorized into two separate groups (as illustrated in FIG. 2):

### I. Copper (Cu(I))—Catalyzed Reactions

This group of reactions mainly comprises, but is not limited to, the Cu(I)-catalyzed Azide-Alkyne (Copper-Catalyzed Azide-Alkyne Cycloaddition, CuAAC).

CuAAC reaction is the most prominent example of click chemistry. An azide-functionalized molecule A reacts with a terminal alkyne-functionalized molecule B thereby forming a stable conjugate A-B via a triazole moiety. The efficiency of a CuAAC reaction strongly depends on the presence of a metal catalyst such as copper (Cu) in the +1 oxidation state (Cu(I)). Different sources and reduction reagents are available. However, the Cu(II) salt CuSO₄ as copper source in combination with ascorbate as a reduction reagent has been recommended for most biomolecule labeling applications. The use of CuAAC reactions in live cells may be hampered by the toxicity of Cu(I) ions. This problem has been overcome by the use of Cu(I) chelating ligands such as tris(3-hydroxypropyltriazolylmethyl)amine (THPTA) that serve a dual purpose: (1) acceleration of the CuAAC reaction by maintaining the Cu(I) oxidation state and (2) protection of the biomolecule from oxidative damage.

### II. Copper Free Reactions

This group of reactions mainly comprises, but is not limited to, the following reactions:

a. Strain-Promoted Azide-Alkyne (Strain-Promoted Azide-Alkyne Cycloaddition, SPAAC)

SPAAC reaction is a non-toxic labeling method. It relies on the use of strained cyclooctynes that possess a remarkably decreased activation energy in contrast to terminal Alkynes and thus do not require an exogenous catalyst. A number of structurally varied cyclooctyne derivatives (e.g. DIFO, BCN, DIBAC, DIBO, ADIBO) have been developed and they differ in terms of reaction kinetics and hydrophility.

b. Alkene-Tetrazine

The Alkene-Tetrazine reaction is also a non-toxic labeling method that is ideally suited for in vivo cell labeling with high-speed and low concentration applications. A terminal or strained Alkene-functionalized molecule reacts with a Tetrazine-functionalized molecule B forming a stable conjugate A-B via dihydropyrazine moiety. A number of structurally varied alkene (e.g. TCO, vinyl, methylcyclopropene) and tetrazine derivatives (e.g. tetrazine, 6-Methyl-Tetrazine) have been developed and they differ in terms of reaction kinetics and hydrophility.

In an embodiment, the invention relates to the method of labeling a bacterium as defined above, wherein the modified choline comprises at least one reactive group X allowing the binding of the modified choline to the tag molecule, said at least one reactive group X being selected from the reactive groups consisting of an alkene group, an alkyne group, an azide group, a cyclopropenyl group and a diazirine group.

In an embodiment, the invention relates to the method of labeling a bacterium as defined above, wherein the modified choline corresponds to the formula (I):

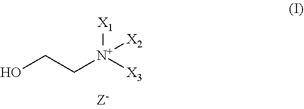

wherein the groups X₁, X₂ and X₃ are selected, independently from each other, from the reactive groups consisting of:
an alkene group

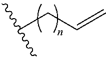

wherein n=0 to 5, in particular n=1, 2, 3, 4 or 5,
an alkyne group

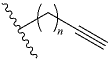

wherein n=0 to 5, in particular n=1, 2, 3, 4 or 5,
an azide group

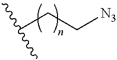

wherein n=0 to 5, in particular n=1, 2, 3, 4 or 5,
a cyclopropenyl group

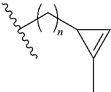

wherein n=0 to 5, in particular n=1, 2, 3, 4 or 5,
a diazirine group

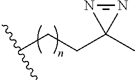

wherein n=0 to 5, in particular n=1, 2, 3, 4 or 5,
and from a methyl group

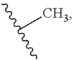

and
at least one, preferably two, more preferably three, of the reactive groups X₁, X₂ and X₃ is different from a methyl group, and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline corresponds to the formula (I):

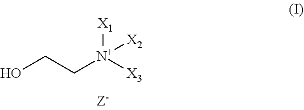

wherein the groups X₁ is an alkene group

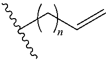

wherein n=0 to 5, in particular n=1, 2, 3, 4 or 5, and X₂ and X₃ are methyl groups

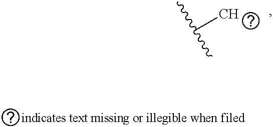

and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline corresponds to the formula (I):

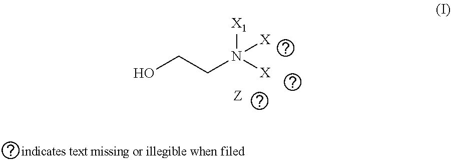

wherein the groups X₁ is an alkyne group

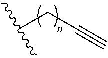

wherein n=0 to 5, in particular n=1, 2, 3, 4 or 5, and X₂ and X₃ are methyl groups

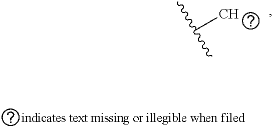

and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline corresponds to the formula (I):

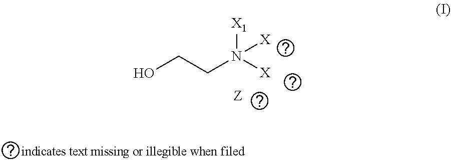

wherein the groups X₁ is an azide group

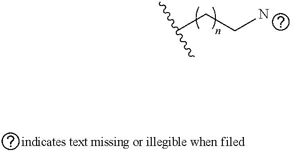

wherein n=0 to 5,
and X₂ and X₃ are methyl groups

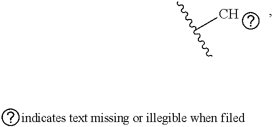

and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline corresponds to the formula (I):

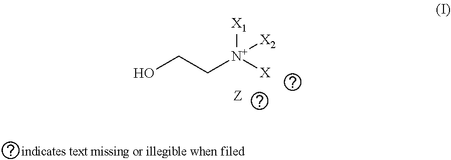

wherein the groups X₁ is a cyclopropenyl group

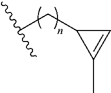

wherein n=0 to 5, in particular n=1, 2, 3, 4 or 5,
and X₂ and X₃ are methyl groups

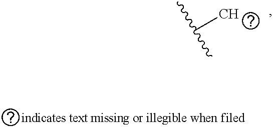

and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline corresponds to the formula (I):

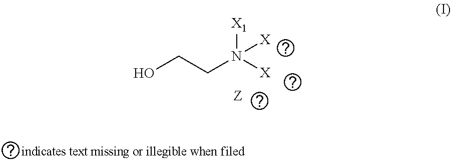

wherein the groups X₁ is a diazirine group

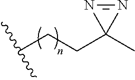

wherein n=0 to 5, in particular n=1, 2, 3, 4 or 5,
and X₂ and X₃ are methyl groups

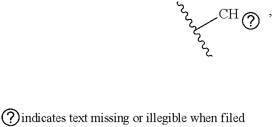

and
Z⁻ is a counterion, preferably selected from F⁻, Cl⁻, Br⁻, I⁻, Tosyl⁻, Mesyl⁻, Triflate⁻, HSO₄⁻ (hydrogen sulphate).

In an embodiment, the invention relates to the method of labeling a bacterium as defined above, wherein the modified choline is selected from the group consisting of:

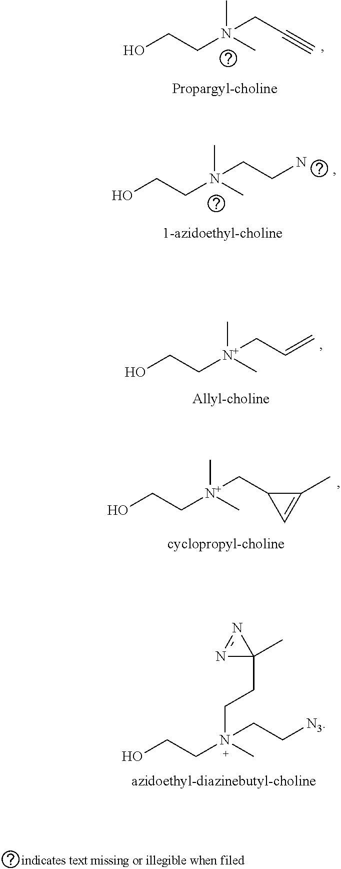

In an embodiment, the invention relates to the method of labeling a bacterium as defined above, wherein the tag molecule is selected from the group consisting of a fluorescent molecule, a luminescent molecule, a radioactive molecule, a biotin molecule or a derivative thereof and an antigen molecule.

In an embodiment, the invention relates to the method of labeling a bacterium as defined above, wherein the tag molecule comprises at least one reactive group Y allowing its binding to the modified choline, said at least one reactive group Y being preferably selected from the group consisting of an alkene group, an alkyne group, an azide group, a cyclopropenyl group, a tetrazine group, a dibenzocyclooctyl (DBCO) group, a dibenzocyclooctine (DIBO) group, a bicyclononine (BCN) group, a Trans-Cyclooctene (TCO) group and a strained Trans-Cyclooctene (sTCO) group.

In an embodiment, the invention relates to the method of labeling a bacterium as defined above, wherein the tag molecule is selected from the group consisting of:

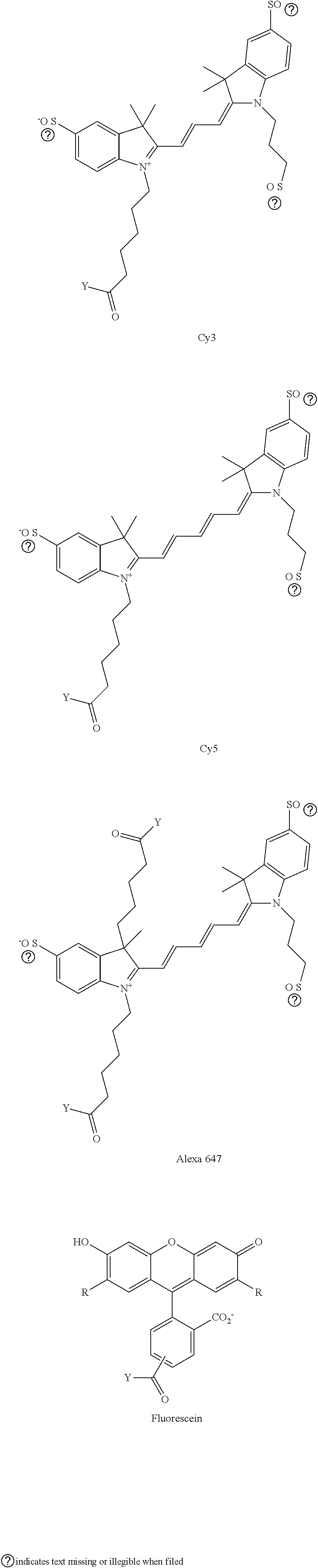

wherein R is H, Alkyl, Aromatic group, OR′, NR′₂, SR′ (R′═H, alkyl),

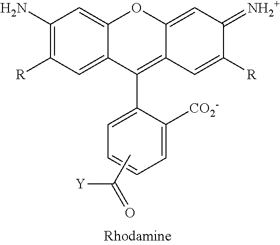

wherein R is H, Alkyl, Aromatic group, OR′, NR′₂, SR′ (R′═H, alkyl),

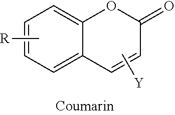

wherein R is H, Alkyl, Aromatic group, OR′, NR′2, SR′ (R′═H, alkyl),

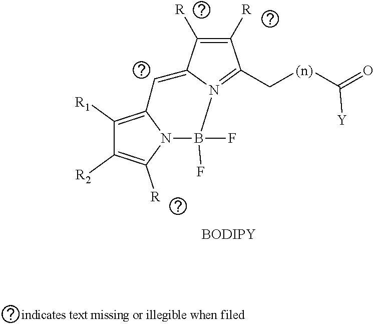

wherein R, R₁, R₂, R₃, R₄ and R₅ are H, Alkyl, Aromatic group, OR′, NR′₂, SR′ (R′═H, alkyl),

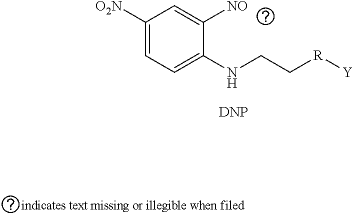

wherein R is Alkyl, Aromatic group,

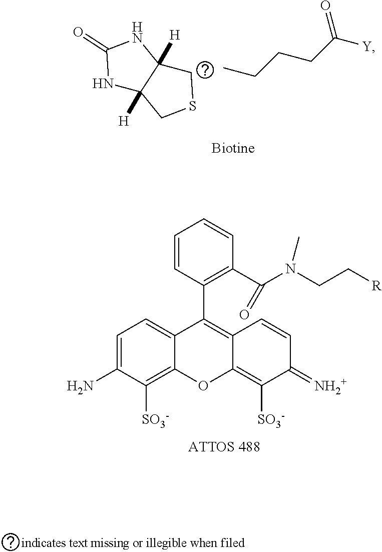

wherein R is Alkyl, Aromatic group,
wherein alkyl groups are composed from 1 to 10 carbons and aromatic groups are benzenic, indolic, furanyl and pyranyl groups,
wherein the reactive group Y is selected from the group consisting of:
an alkene group

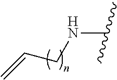

wherein n=0 to 5, in particular n=1, 2, 3, 4 or 5,
an alkyne group

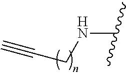

wherein n=0 to 5, in particular n=1, 2, 3, 4 or 5,
an azide group

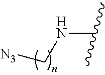

wherein n=0 to 5, in particular n=1, 2, 3, 4 or 5,
a cyclopropenyl group

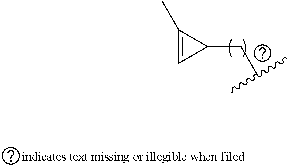

wherein n=0 to 5, in particular n=1, 2, 3, 4 or 5,
a tetrazine group

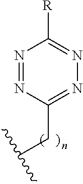

wherein n=0 to 10, in particular n=1, 2, 3, 4, 5, 6, 7, 8, 9 or 10,
a DBCO group

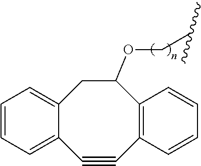

wherein n=0 to 10, in particular n=1, 2, 3, 4, 5, 6, 7, 8, 9 or 10,
a DIBO group

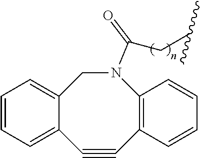

wherein n=0 to 10, in particular n=1, 2, 3, 4, 5, 6, 7, 8, 9 or 10,
a BCN group

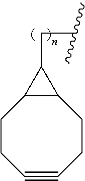

wherein n=0 to 10, in particular n=1, 2, 3, 4, 5, 6, 7, 8, 9 or 10,
a TCO group

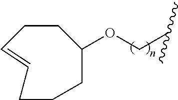

wherein n=0 to 10, in particular n=1, 2, 3, 4, 5, 6, 7, 8, 9 or 10,
a sTCO group

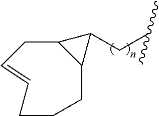

wherein n=0 to 10, in particular n=1, 2, 3, 4, 5, 6, 7, 8, 9 or 10.

In an embodiment, the invention relates to the method of labeling a bacterium as defined above, wherein the tag molecule is fluorescent (such as Fluorescein, Rhodamine, Bodipy, . . . ) and contains a clickable function (such as alkyne, azide, DIBO, tetrazine, . . . ).

In an embodiment, the invention relates to the method of labeling a bacterium as defined above, wherein the tag molecule is selected from the group consisting of:
3-Azido-7-(diethylamino)-2H-chromen-2-one and
7-nitro-N-(prop-2-yn-1-yl)benzo[c][1,2,5]oxadiazol-4-amine.

In the invention, at least one reactive group X of the modified choline bound to the TA in the cell wall of the bacterium will react with one reactive group Y of the tag molecule via a bioorthogonal reaction, in particular by a Click chemistry reaction, to form a conjugate and to allow the labeling of the bacterium.

In an embodiment, the invention relates to the method of labeling a bacterium as defined above, wherein the reactive group X of the modified choline and the reactive group Y of tag molecule are respectively selected from the clickable couples recited in Table 1.

TABLE 1
Clickable couples of reactive groups X of the modified
choline/reactive groups Y of the tag molecule.

| Reactive group X (modified choline) | Reactive group Y (tag molecule) |
|---|---|
| Alkene | Tetrazine |
| Alkyne | Azide, Tetrazine |
| Azide | DIBO, DBCO, BCN |
| Cyclopropenyl | Tetrazine |

In the invention, the medium used to incubate the bacterium is selected according to the bacterium to label. The appropriate medium can be selected and/or adapted by one skilled in the art from commercial media or from routinely used media.

In the case of S. pneumoniae, a C-medium is preferably used. The composition of the C-medium is given in Lacks S, Hotchkiss R D. 1960 (A study of the genetic material determining an enzyme in Pneumococcus. Biochem. Biophys. Acta, 39:508-518).

For the incubation of the bacterium with the modified choline, the modified choline can be present in the culture medium at various concentrations.

In an embodiment, the invention relates to the method of labelling a bacterium as defined above, wherein, at step (i), the modified choline is present at a concentration of 1 μg/ml to 1 mg/ml, preferably 1 to 100 μg/ml, more preferably 1 to 10 μg/ml, in the culture medium.

As a non-limitative example, the modified choline can be present at a concentration of 1, 2, 3, 4, 5, 6, 7, 8, 9, 10, 20, 50, 100, 200, 500 or 1 000 μg/ml in the culture medium.

For the labelling step, the bacterium can be incubated in presence of a Copper ligand, such as the THTPA.

The THPTA ligand binds Cu(I), blocking the bioavailability of Cu(I) and ameliorating the potential toxic effects while maintaining the catalytic effectiveness in Click conjugations. The THPTA ligand is used to label living cells with high efficiency while maintaining cell viability.

The time of incubation of the bacterium with the modified choline can vary from few seconds to few hours.

In an embodiment, the invention relates to the method of labelling a bacterium as defined above, wherein, at step (i), the bacterium is incubated with the modified choline for 15 sec to 3 hours or more, preferably for 15 sec to 30 min, even more preferably for 15 to 60 sec.

As a non-limitative example, the time of incubation with the modified choline can be 15 sec, 30 sec, 1 min, 2 min, 10 min, 30 min, 1 h, 2 h or 3 h.

In an embodiment, the invention relates to the method of labelling a bacterium as defined above, wherein, at step (ii), a Copper ligand, such as THTPA, is present in the culture medium.

In an embodiment, the invention relates to the method of labelling a bacterium as defined above, wherein, at step (ii), the bacterium is incubated with the tag molecule for 1 min to 3 hours or more, preferably for 1 min to 30 min, even more preferably for 1 min to 5 min.

As a non-limitative example, the time of incubation with the tag molecule can be 1 min, 2 min, 10 min, 30 min, 1 h, 2 h or 3 h.

In an embodiment, the invention relates to the method of labelling a bacterium as defined above, wherein, at step (i), the bacterium is incubated with the modified choline for 15 sec, preferably with 1-azidoethyl-choline, and at step (ii), the bacterium is incubated with the tag molecule for 5 min, preferably with a tag molecule carrying a DIBO function such as DIBO-ATTOS 488.

The present invention thus allows a fast labelling and detection of the target bacteria.

In an embodiment, the invention relates to the method of labelling a bacterium as defined above, wherein said steps (i) and (ii) are performed simultaneously.

As shown in the examples, when the metabolization of the modified choline (step i) is significantly faster than the click reaction with the tag molecule (step ii), both reagents can be added simultaneously, making the method comparable to direct metabolic approaches. This approach can be referred to as the “one-pot” approach.

In particular, the invention relates to the method of labelling a bacterium as defined above, wherein said steps (i) and (ii) are performed simultaneously and the modified choline reacts with the tag molecule via a strain promoted azide alkyne cycloaddition (SPAAC). This embodiment combines the rapid kinetics of a SPAAC reaction with the speed of a biological process.

In particular, the invention relates to the method of labelling a bacterium as defined above, wherein said steps (i) and (ii) are performed simultaneously and wherein the modified choline carries an azide function and the tag molecule carries a DIBO, preferably a dibenzoannulated cyclooctyne DIBO.

In an embodiment, the invention relates to the method of labeling a bacterium as defined above, that further comprises a step (iii) of detection and/or quantification of the bacterium by detecting and/or quantifying the tag molecule bound to the bacterium.

The step of detection and/or quantification of the bacterium can be achieved by various techniques depending on the Tag molecule that has been used for the labeling. These routine techniques (such as epifluorescence, tomography, . . . ) are well-known by one skilled in the relevant art.

The labeling method of the invention facilitates the detection of the bacterium since it allows a high density labeling, with a grafting level of more than 70%, compared to other labeling techniques such as incorporation of D-amino acids (≈2-3%). The grafting level can be determined by various methods well known to the one skilled in the art, such as HPLC analyses (Kuru et al., 2012. In situ probing of newly synthesized peptidoglycan in live bacteria with fluorescent D-amino acids. Angew. Chem. Int. Ed. Engl. 51, 12519-12523).

In a preferred embodiment, the invention relates to a method of labeling a bacterium that is able to metabolize choline, preferably S. pneumoniae, said method comprising:
a step (i) of incubating the bacterium in a culture medium containing a modified choline during which the modified choline is metabolized by the bacterium and covalently bound to the teichoic acid present in the cell wall of the bacterium,
a step (ii) of contacting the bacterium with a tag molecule to generate a binding reaction between the modified choline bound to the teichoic acid present in the cell wall of the bacterium and the tag molecule, said binding reaction being preferably made by a Click chemistry reaction, and
optionally, a step (iii) of detection and/or quantification of the bacterium by detecting and/or quantifying the tag molecule bound to the bacterium.

In a preferred embodiment, the invention relates to a method of labeling a bacterium that is able to metabolize choline, preferably S. pneumoniae, said method comprising:
a step (i) of incubating the bacterium in a culture medium containing a modified choline at a concentration of 1 μg/ml to 1 mg/ml, preferably of 1 μg/ml to 10 μg/ml, for 15 sec to 30 min, preferably for 15 to 60 sec, during which the modified choline is metabolized by the bacterium and covalently bound to the teichoic acid present in the cell wall of the bacterium,
a step (ii) of contacting the bacterium with a tag molecule for 1 min to 30 min, preferably for 1 min to 5 min, to generate a binding reaction between the modified choline bound to the teichoic acid present in the cell wall of the bacterium and the tag molecule, said binding reaction being preferably made by a Click chemistry reaction, and
optionally, a step (iii) of detection and/or quantification of the bacterium by detecting and/or quantifying the tag molecule bound to the bacterium.

In a preferred protocol, a bacterium S. pneumoniae is grown, in C-medium in presence of choline, preferably at 6 μg/ml up to an absorbance of 0.3, then the culture is centrifuged and the bacterium is washed with C-medium without choline and concentrated, preferably by a factor 100. Then, 50 μl of the bacterium in suspension is incubated for 15 to 60 sec in C-medium containing 6 μg/ml of azide-choline at 37° C., then 25 μM of DIBO-ATTOS 488 is added in the C-medium, then the culture is incubated at 37° C. for 5 min. The reaction is stopped by three washes with 1 ml of cold PBS. The bacterium is then re-suspended in 20-40 μl of PBS and observed immediately by fluorescence microscopy.

### Bio-Imagery

In another aspect, the invention relates to an in vitro method of tracking a bacterium by bio-imagery comprising a step whereby the bacterium is labeled by using the labeling method defined above with a tag molecule allowing the follow-up, preferably in real-time, of the bacterium.

In the field of bio-imaging, the metabolites to be incorporated are all quite expensive to produce (e.g. azidio-functionalized L-fucose, KDO sugar, ketone bearing derivatives MurNAc-pentapeptide, GlcNAc precursor, modified tripeptideL-alanyl-g-D-glutamyl-L-Lysine, fluorescent D-amino acids). Thus, the method of the invention has the advantage to be more affordable because the modified cholines can be produced in on-step from 2-(dimethylamino)ethan-1-ol.

For radiolabeling, radiolabeled metabolites like ¹⁸F or ¹⁴C sugar are commonly used but are expensive and complicated to synthesize and to purify. The method of the invention is simpler because it can use Na¹²⁵I as radiolabeling reagent.

An illustration of the method of tracking is shown in FIG. 3.

### Diagnosis

In another aspect, the invention relates to an in vitro method for the diagnosis of a bacterial infection from a biological sample of a patient comprising a step whereby the bacterium responsible of the infection is labeled by using the labeling method defined above with a tag molecule allowing the detection of the bacterium.

The method of labeling of the invention can be used to detect the bacteria that are present in a biological sample or the bacteria that have been previously isolated from a biological sample.

The detection of a tagged bacterium is indicative of an infection by said bacterium.

An illustration of the diagnostic method is shown in FIG. 4.

### Vaccination

In another aspect, the invention relates to a method for the preparation of a vaccine composition containing a bacterium or fragments thereof, comprising a step whereby the bacterium is in vitro labeled by using the labeling method defined above with an antigen, said antigen being bound to the bacterium or to the fragments thereof.

The method of the invention allows to graft a high-number of antigens at the surface of the bacterium and, advantageously, onto the cell wall of the bacterium, which is generally used as a shield by the bacterium to escape or to protect itself from the host immune response. Since the method of labeling allows a high density labeling, this feature is used to turn the bacterium into a multivalent antigen-presenting reagent.

In the invention, the term “antigen” refers to any molecule or compound that induces or enhances an immune response in the host to whom it is administered. In particular, the term antigen covers epitopes that are specifically recognized by antibodies and receptors of the immune agents, such as T-cells, B-cells, NK-cells, . . . .

In an embodiment, the invention relates to a method for the preparation of a vaccine composition as defined above, which further comprises a step wherein, after labeling, the bacterium is killed, for example by heat treatment or grinding.

In an embodiment, the invention relates to a method for the preparation of a vaccine composition as defined above, which further comprises a step wherein, after labeling, the content of the bacterium is emptied to generate tagged-sacculi, i.e. the exoskeleton of the bacterium.

The vaccine composition obtained by the method of the invention can contain live bacteria, dead bacteria, emptied bacteria or fragments of bacteria.

An illustration of the method to prepare a vaccine composition is shown in FIG. 5.

### Bio-Materials

In another aspect, the invention relates to an in vitro method for the preparation of a bio-material comprising:
a step of labeling a first population of bacteria with a first modified choline by using the labeling method as defined above,
a step of labeling a second population of bacteria with a second modified choline by using the labeling method as defined above,
   the first modified choline used to label the first population and the second modified choline used to label the second population being respectively chosen to cross-react by Click chemistry between each other, and
then, a step of binding the first population with the second population by Click chemistry to form a bio-material.

In the invention, the bio-material can be assimilated to a prokaryote tissue composed by inter-connected bacteria, or fragments of bacteria, linked to each other.

The method of labeling of the invention can thus be used to create bonds between populations of bacteria that have metabolized cross-reacting modified cholines.

In an embodiment, the invention relates to a method for the preparation of a bio-material as defined above that further comprises a step of killing the bacteria, while preserving the cell structure, i.e. the exoskeleton, of the bacteria and the links operated between bacteria.

An illustration of the method to produce bio-materials is shown in FIG. 6.

### Identification of Inhibitors of the Cell Wall Synthesis

In another aspect, the invention relates to an in vitro method of identifying an agent that inhibits the bacterial cell wall synthesis, said method comprising:
a step of contacting a bacterium with a test agent,
a step of labeling the bacterium using the labeling method defined above,
   wherein a test agent that inhibits the labeling of the bacterium is considered a candidate agent for inhibiting the cell wall synthesis of the bacterium.

In particular, this method can be transposed for a high-throughput screening method to screen inhibitors of the peptidoglycane synthesis. The decreased in intensity (e.g. fluorescence) of the signal is correlated to drug susceptibility.

### Kit

In an aspect, the invention relates to a kit to label a bacterium comprising:
a modified choline as defined above,
a tag molecule as defined above, and
a culture medium allowing the growth of the bacterium.

The following figures and examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### FIGURE LEGENDS

FIG. 1. Illustration of the structure of TA from S. pneumoniae (adapted from Gisch et al., J. Biol. Chem., 288:15654, 2013)

FIG. 2. Illustration of the Copper-dependent and Copper-independent Click chemistry pathways.

FIG. 3. Bacteria coated by alkyne or azide groups are labeled in one step to provide a fluorophore (Pathway A) or a radio-tracer (pathway B).

FIG. 4. Illustration of the use of the labeling method of the invention for diagnostic applications.

FIG. 5. Illustration of the use of the labeling method of the invention to coat bacteria cell surface with immune-stimulating epitopes.

FIG. 6. Illustration of the covalent interlocking of bacteria to form prokaryote tissues and new bio-materials.

FIG. 7. A. Metabolization of modified cholines (S. pneumoniae growth with 10 μg/mL). B. Exogenous GFP-LytA displays similar binding pattern on cells grown in presence of propargyl-choline and of normal choline (fluorescent and phase contrast images are shown).

FIG. 8. NMR analysis of the propargyl-choline incorporation. Sections of ³¹P NMR (δ_{P} 2.5-(−1.0)) and ¹H NMR (water suppressed; δ_{H} 6.0-0.0)) spectra of PGN-WTA preparations after LytA treatment, isolated from S. pneumoniae grown in the presence of (i) normal choline (and performed bioorthogonal reaction with 3-azido-7-(diethylamino)-2H-chromen-2-one (2)), (ii) propargyl-choline and (iii) propargyl-choline (and performed bioorthogonal reaction with 2). Incorporation of propargyl-choline in WTA is proven by the occurrence of the additional signals in panels (ii) and (iii).

FIG. 9. Detection of metabolically incorporated propargyl-choline in S. pneumoniae fixed cells before bioorthogonal reaction with coumarin. Numbers (i) to (iii) refer to different stages of division. Phase contrast and fluorescence images in absence (A) or presence (B) of propargyl-choline are shown. Scale bar=1 μm

FIG. 10. TA metabolic labeling on pneumococcal live cells. A. Detection of metabolically incorporated propargyl-choline in S. pneumoniae living cells after bioorthogonal reaction. (i) and (ii) refer to two different stages of division. Membrane and septal TA labeling are indicated by black and white arrow heads, respectively. Phase contrast and fluorescence images in the presence (left panel) or absence of propargyl-choline (right panel) are shown. Scale bar=1 μm. B. Electron micrographs of pneumococcal cells at division stages (i) and (ii) as shown in A. C. Demograph of a pneucococcal cell population grown in the presence of propargyl-choline and submitted to the bioorthogonal reaction shows the mid-cell positioning of TA.

FIG. 11. TA metabolic labeling on live cells after 30-min pulse of propargyl-choline. A. Numbers (i) to (iii) refer to the different stages of division. Phase contrast and fluorescence images are shown. Scale bar=1 μm. B. Demograph of pneucococcal cell population grown in the presence of propargyl-choline for 30 min and labeled with propargyl-choline-coumarin showing the mid-cell positioning of TA.

FIG. 12. Specific detection of metabolically incorporated propargyl-choline in live S. pneumoniae cells. Cultures of E. coli, B. subtilis and P. aeruginosa were performed as for S. pneumoniae in the presence (+propargyl-Cho) or in the absence (+Cho) of propargyl-choline. Cultures were mixed as indicated on the left hand side of the figure and processed for biorthogonal reaction.

FIG. 13. Short pulse. The bacterium is incubated for 15 seconds (left) or 60 seconds (right) with the the azide-choline, and then incubated for 5 minutes with DIBO-ATTOS 488.

FIG. 14. Two-step and direct labeling of TA and PG in S. pneumoniae, respectively. PG is depicted by the joined ellipses and TA by black bars. Purple arrows outline the metabolization of propargyl-choline from its import to its exposure at the cell surface attached to TA. The arrow describes the “click” reaction between the azido- and alkyne groups or the exchange of the distal D-Ala of the PG for FDAA catalyzed by PBPs.

FIG. 15. Growth curve of S. pneumoniae R800. In C-medium without choline (*), with 30 μM azidocholine (†) or choline (‡).

FIG. 16. Labeling of TA of S. pneumoniae. Demographs show the fluorescence intensity along individual cells ordered by size. (a) Bacteria grown with 30 μM azido-choline for 4 hours, incubated with 25 μM DIBO Alexa Fluor™ 594 for 5 min prior to imaging. (b) Attempt at pulse labeling by incubation for 5 min with 30 μM azido-choline and subsequent addition of 25 μM DIBO reagent for 5 min. (c) Pulse labeling of TA by “one-pot” simultaneous addition of 30 μM azido-choline and 25 μM reagent for 5 min.

FIG. 17. (a) Cells were treated for 5 min with 30 μM choline and 25 μM DIBO Alexa Fluor™ 488 at 37° C. prior to imaging. (b) Azido-choline and DIBO Alexa Fluor™ 488 were incubated overnight together prior to incubation for 5 min with cells at 30 μM and 25 μM, respectively, in C-medium without choline.

FIG. 18. Scheme of the two-step “one-pot” labeling of pneumococcal TA. Azido-choline 1 and DIBO Alexa Fluor™ 2 are added simultaneously to growing cells. The different rates of the azido-choline metabolization and SPAAC reaction ensure that both reagents can be added together for adequate labeling in short pulses.

FIG. 19. Bio-orthogonal labeling of WTA and LTA of S. pneumoniae. Bacteria were grown in choline free C-medium supplemented with 30 μM choline for 2 hours. TA were then labeled by incubation with 30 μM azido-choline and 25 μM DIBO Alexa Fluor™ 594 for 5 min. (a) Sacculi obtained from pulse-labeled cells by boiling for 45 min in 4% SDS. (b) Spheroplasts obtained from pulse-labeled cells by digesting cells with lysozyme, mutanolysin and LytA for 60 min at 37° C. and overnight incubation at 4° C.

FIG. 20. Geography of pneumococcal cell surface. Equators (E) and division sites (D) are co-localized at the onset of a new division. Parental hemispheres are in gray, daughter hemispheres are in white. The arrows indicate that the division sites are “moving” away from the duplicated equators.

FIG. 21. Pulse-chase labeling of TA in S. pneumoniae expressing fluorescent FtsZ-mKate. (a) TA were labeled by a 5 min pulse of 30 μM azido-choline and 25 μM DIBO Alexa Fluor™ 488 then chased by diluting in medium with 30 μM choline. (b) Demographs showing the distribution of pulse-labeled TA and FtsZ localization.

FIG. 22. Fluorescent time lapse microscopy of pulse labeled TAs of S. pneumoniae on agarose pad. (a) Overlay of bright field and 5 min labeled-TA of wild type S. pneumoniae grown on agarose pad for 50 min. (b) Overlay of bright field, 5 min labeled-TAs and FtsZ of S. pneumoniae cells expressing FtsZ-mKate grown on agarose pad for 70 min.

FIG. 23. Comparison of the pulse labeling of TA and PG, which were labeled for 5 min with 30 μM azido-choline, 25 μM DIBO Alexa Fluor™ 594 and 500 μM HADA (a). (b) Demographs showing the localization of newly inserted TA and PG. Fluorescence intensities of the longest cells are enlarged to emphasize the different patterns. (c) TA (upper picture), and PG labeling (lower picture) of a representative cell. The longitudinal axis is highlighted by a line. Outlines of the fluorescent intensity over the cell length are shown with the peak intensities used to calculate the ratio division site (D)/equators (E). (d) The ratio of fluorescence intensities at sites D/E as a function of cell length. (e) The ratio of fluorescence intensities at sites D/E of labeled TA vs that of PG.

FIG. 24. Pulse-chase labeling of TAs and PG. (a) TA and PG were labeled by a 5 min pulse of 30 μM azido-choline, 25 μM DIBO Alexa Fluor™ 488 and 500 μM HADA in C-medium at 37° C. then chased by diluting in C-medium supplemented with 30 μM of choline. Fluorescence images of labeled TA, PG and merged images with the bright field are shown. (b) Demographs showing the distribution of pulse-labeled TA and PG in the population during the chase.

FIG. 25. TA and PG labeling of S. pneumoniae mutant strains. TA and PG were labeled by a 5 min pulse of 30 μM azido-choline, 25 μM DIBO Alexa Fluor™ 488 and 500 μM HADA at 37° C. (a) Fluorescence images of TAs, PGs and the merged images with bright field are shown. (b) Demographs showing the localization of newly incorporated TA and synthesized PG for each mutant strain.

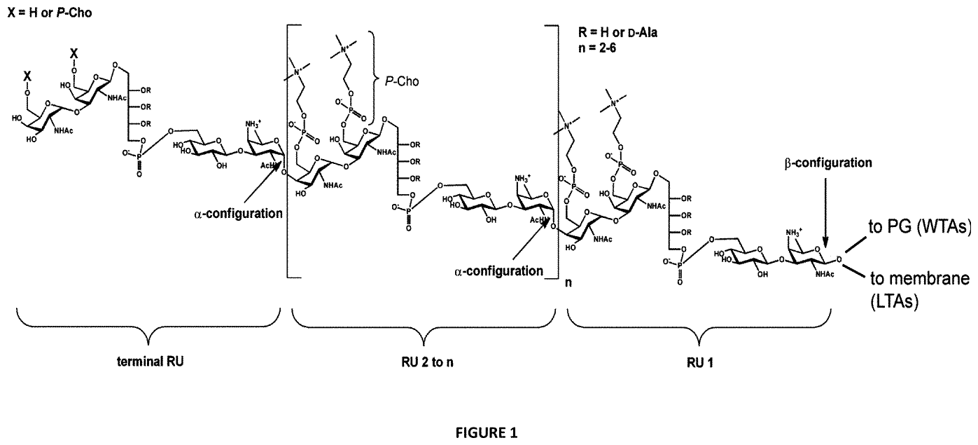

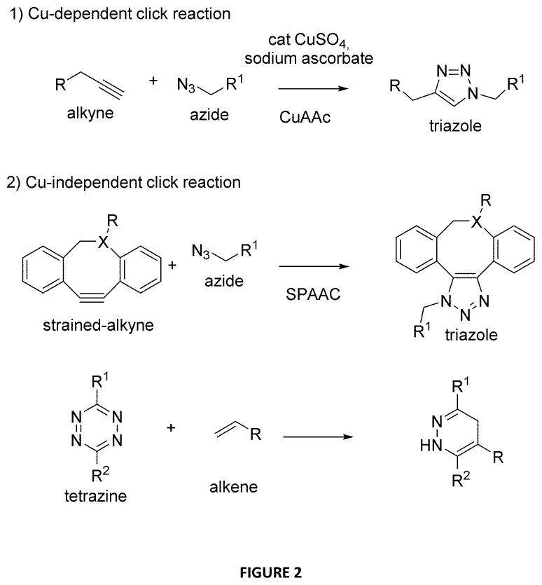

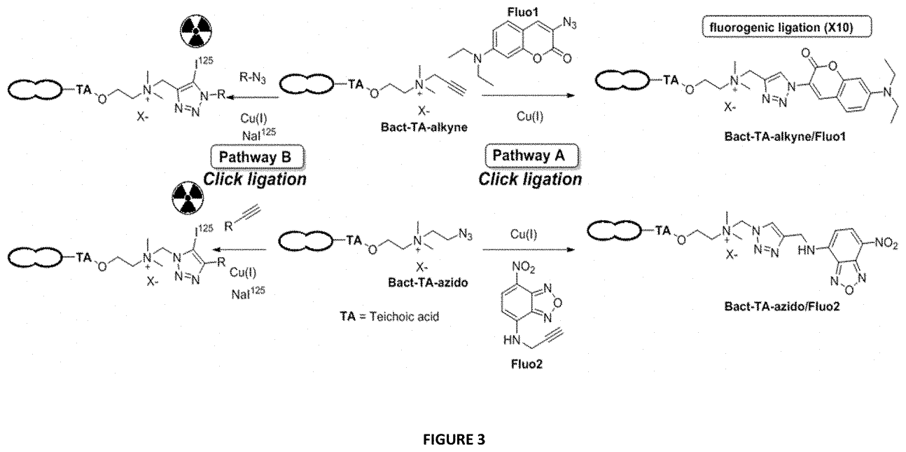

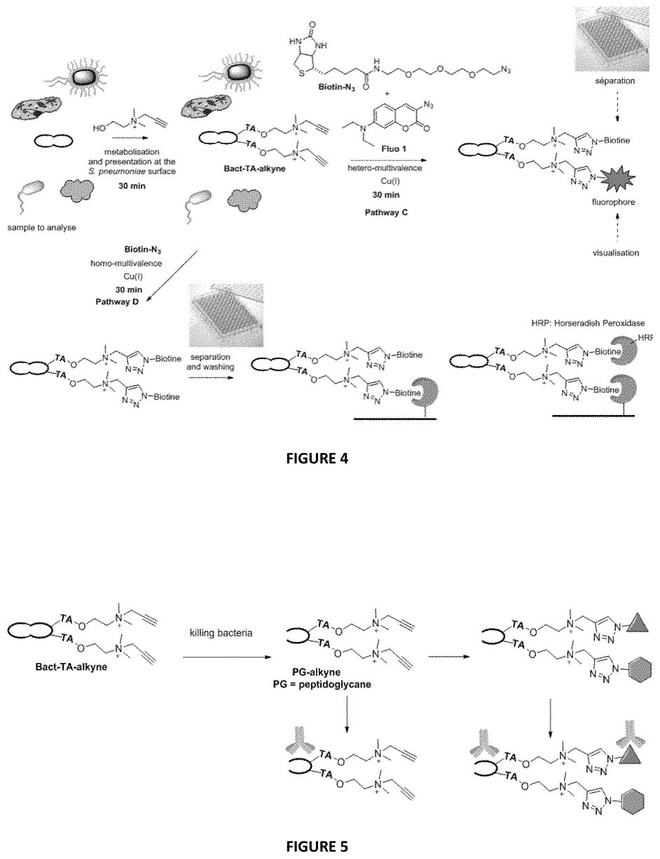

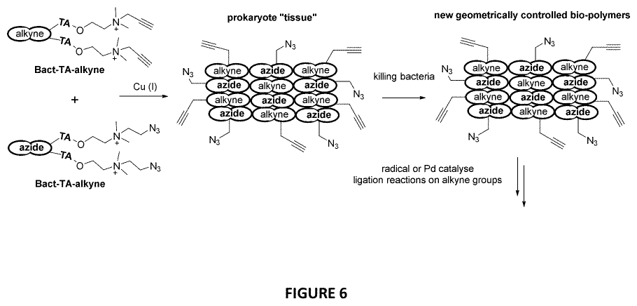

### EXAMPLES

### Materials and Methods

### Bacterial Growth Conditions

Liquid cultures of the unencapsulated pneumococcal strain R6 were grown at 37° C.-5% CO₂ in a chemically defined medium (C-medium) supplemented with 4 μg/ml choline (Cmed-choline). Contrary to the original composition, the C-medium used here did not contain neither yeast extract nor albumin. Cells were harvested by centrifugation at 3,320 g for 10 min, washed three times with C-medium without choline, concentrated to OD_{600 nm} of 2 and stored at −80° C. as aliquots containing 15% glycerol (v/v).

For bioorthogonal reactions, 10 ml of Cmed-choline and 10 ml of C-medium containing 4 μg/ml of propargyl-choline were inoculated at OD_{600 nm} of 0.05 with aliquots of cells conditioned in C-medium as described above. The growth was pursued at 37° C.-5% CO₂ for 3 hours until OD_{600 nm} of 0.2-0.25 was reached, which corresponds to the early exponential growth phase. The cells were pelleted by centrifugation at 3,320 g for 10 min and subsequently incubated with 500 μl of 2% choline chloride (w/v) for 10 min at room temperature (RT) to remove the Choline-Binding Proteins (CBPs) that bind to choline residues. In the case of choline-alkyl residues, the presence of CBPs possibly impair the labeling of those molecules by the fluorescent azide reporter. The cells were washed twice with PBS (1 min centrifugation at 4,500 g) and resuspended in 400 μl of PBS. A volume of 100 μl was used for each bioorthogonal reaction. In pulse experiments, cells were grown in Cmed-choline for 3 h, washed twice in PBS, resuspended in C-medium containing 4 μg/ml of propargylcholine or 1-azidoethyl-choline and incubated at 37° C.-5% CO₂ for 30 min before proceeding to the labeling.

Escherichia coli, Bacillus subtilis and P. aeruginosa growth conditions in C-medium supplemented with both forms of choline were tested before conducting the click reactions with the same protocol as the one developed for Streptococcus pneumoniae.

### Copper Catalyzed Click Chemistry

Labeling was performed on cells grown in presence of choline and choline-alkyl. A volume of 100 μl of cell suspension prepared as described above was incubated with the following reagents, which final concentration is indicated: coumarin (1 mM), ascorbic acid (1 mM), Copper (II) sulfate (50 μM), THPTA (tris(3-hydroxypropyltriazolylmethyl)amine) (300 μM) for 30 min at RT, under mild agitation and protected from the light. Labeled cells were washed twice with PBS and resuspended in PBS for microscopy observation.

Cell fixation was performed after culture harvest. Cells from 10 ml culture were washed twice with PBS, resuspended in 500 μl of 4% (w/v) paraformaldehyde for 30 min at RT followed by a 2 h-incubation at 4° C. After two washes with PBS, cells were resuspended in 400 μl of PBS and aliquots of 100 μl were used for the click reaction by adding the coumarin (1 mM), ascorbic acid (1 mM) and Copper (II) sulfate (100 μM). The labeling proceeded for 16 h at RT under mild agitation and protected from the light. Labeled cells were washed twice with PBS and resuspended in PBS before microscopy observation.

### Fluorescence Microscopy and Image Analysis

Pneumococcal cells were transferred to microscope slides and observed using an Olympus BX61 optical microscope equipped with a UPFLN 100× O-2PH/1.3 objective and a QImaging Retiga-SRV 1394 cooled charge-coupled device camera. Image acquisition and analysis were performed using the software packages Volocity and open-source Oufti, respectively and processed with Adobe Photoshop CS5. Cell population demographs were constructed by Oufti which integrates the signal values in each cell. The cells are then sorted by their length value and the fluorescence values are plotted as a heat map.

### Extraction and Isolation of LTA

Pneumococcal cells were resuspended in citrate buffer (50 mM, pH 4.7) and disrupted three times by French press (Constant Cell Disruption System, Serial No. 1020) at 10° C. at a pressure of 20 kPSI. SDS was added to a final concentration of 4% to the combined supernatants. The solution was incubated for 30 min at 100° C. and was stirred afterwards overnight at room temperature. The solution was centrifuged at 30,000×g for 15 min at 4° C. The pellet was washed four times with citrate buffer using the centrifugation conditions as above. The combined LTA-containing supernatants and the resulting sediment, containing the crude PGN-WTA complex, were lyophilized separately. The resulting solids were both washed five times with ethanol (centrifugation: 20 min, 20° C., 10,650×g) to remove SDS and lyophilized (leading to pellet A containing LTA and pellet B containing the PGN-WTA complex). For LTA isolation, pellet A was resuspended in citrate buffer and extracted with an equal volume of butan-1-ol (Merck) at room temperature under vigorous stirring. The phases were separated by centrifugation at 4,000×g for 15 min at 4° C. The aqueous phase (containing LTA) was collected, and the extraction procedure was repeated twice with the organic phase plus interphase. The combined aqueous phases were lyophilized and subsequently dialyzed for 5 days at 4° C. against 50 mM ammonium acetate buffer (pH 4.7; 3.5 kDa cut-off membrane); the buffer was changed every 24 h. The resulting crude LTA was purified further by hydrophobic interaction chromatography (HIC) performed on a HiPrep Octyl-Sepharose column (GE Healthcare; 16×100 mm, bed volume 20 ml). The crude LTA material was dissolved in as little starting buffer (15% propan-1-ol (Roth) in 0.1 M ammonium acetate (pH 4.7)) as possible and centrifuged at 13,000×g for 5 min at room temperature and the resulting supernatant was lyophilized. The LTA-containing pellet was dissolved in the HIC start buffer at a concentration of 30 mg/ml and purified by HIC using a linear gradient from 15% to 60% propan-1-ol (Roth) in 0.1 M ammonium acetate (pH 4.7). LTA-containing fractions were identified by a photometric phosphate test. The phosphate-containing fractions were combined, lyophilized and washed with water upon freeze-drying to remove residual buffer.

### Extraction and Isolation of WTA

Pellet B (containing the crude PGN-WTA complex), which arose during LTA isolation, was resuspended at a concentration of 10 mg/ml in 100 mM Tris-HCl (pH 7.5) containing 20 mM MgSO₄. DNase A and RNase I were added to final concentrations of 10 and 50 μg/ml, respectively. The suspension was stirred for 2 h at 37° C. Subsequently, 10 mM CaCl₂ and trypsin (100 μg/ml) were added and the stirring was continued overnight at 37° C. SDS at a final concentration of 1% was added, and the mixture was incubated for 15 min at 80° C. to inactivate the enzymes. The cell wall was recovered by centrifugation for 45 min at 130,000×g at 37° C. The resulting pellet was resuspended in 0.8 ml 8 M LiCl per 1 ml initially used Tris-HCl solution and incubated for 15 min at 37° C. After another centrifugation using the same conditions as above, the pellet was resuspended in 1 ml 10 mM ethylenediaminetetraacetic acid (EDTA, pH 7.0) per ml of the Tris-HCl solution used initially and this sample was incubated at 37° C. for 15 min. The pellet was washed twice with water. Finally, the pellet was resuspended in 2 to 4 ml of water and lyophilized, yielding the purified PGN-WTA complex. To remove all amino acids from the PGN, the PGN-WTA complex was dissolved in 50 mM Tris-HCl (pH 7.0; 10 mg/ml) and treated with the pneumococcal LytA amidase. Recombinant His-tagged LytA amidase (1 mg/10 μg LytA) was added in three aliquots after 0, 24 and 48 h for a total period of incubation of 72 h at 37° C. Subsequently, the enzyme was inactivated by boiling for 5 min at 100° C. After centrifugation (25,000×g, 15 min, 20° C.) the supernatant was collected and lyophilized. The crude LytA-treated PGN-WTA complex was further purified by GPC on a Bio-Gel P-30 (45-90 μm, BioRad; column size: 1.5×120 cm; buffer: 150 mM ammonium acetate (pH 4.7)) column.

### Example 1. Metabolic Incorporation of Modified Choline

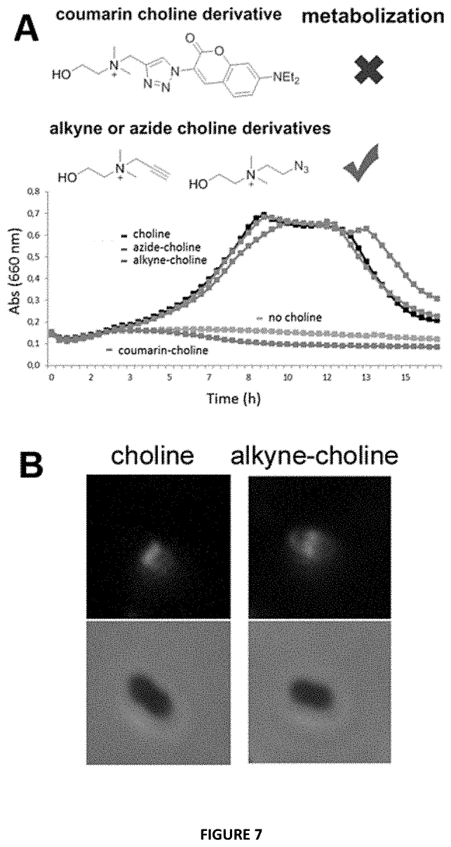

The choline dependency for pneumococcal growth was exploited to validate the metabolic incorporation of modified cholines. Propargyl-choline was evaluated as well as its corresponding fluorescent analogue (i.e. propargyl-choline-coumarin) obtained by the click reaction between propargyl-choline and coumarin. One interesting aspect of the coumarin comes from its fluorogenic property once coupled with alkyne that amplifies the fluorescence signal to reduce the background interference. Nonpathogenic (unencapsulated) pneumococcal R6 strain was cultured in C-medium containing propargyl-choline-coumarin or normal choline. Comparable growth rates were observed in the presence of normal choline and of propargyl-choline indicating a good metabolisation of the latter compound (FIG. 7).

### Example 2. Analytical Characterization and Quantification of TA Decorated by Modified Choline

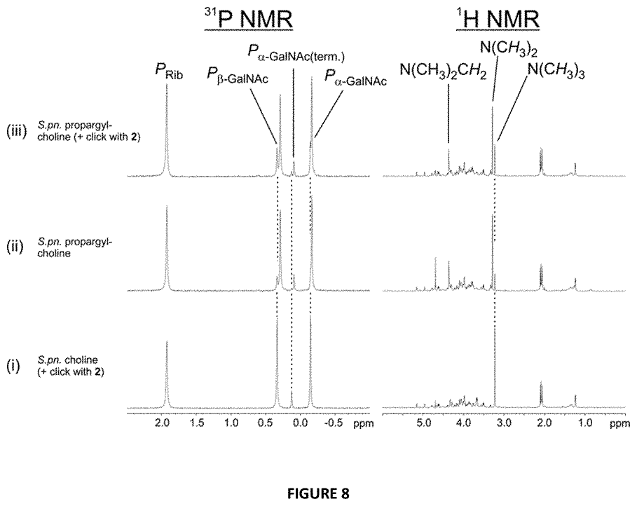

Pneumococcal cultures grown in presence of propargyl-choline or normal choline were processed to extract LTA. NMR analysis showed that propargyl-choline has been integrated in LTA (FIG. 8).

### Example 3. Detection of Metabolically Incorporated Modified Choline

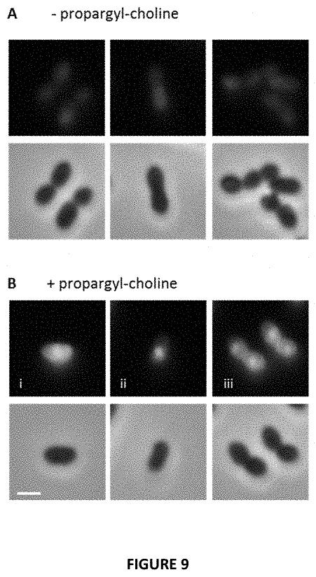

To prevent any modification of the structure of TA and/or their eventual re-localization at the cell surface during the chemical labeling, a preliminary study was performed with cell containing propargyl-choline fixed prior the bioorthogonal reaction. Fluorescence was specifically detected on cells grown in the presence of propargyl-choline when compared with cells grown in the presence of normal choline (FIG. 9). Bright fluorescent spots were observed at the mid-cell position in early (panels i and ii) and late division stages (panel iii).

### Example 4. Metabolic Labeling on Pneumococcal Live Cells

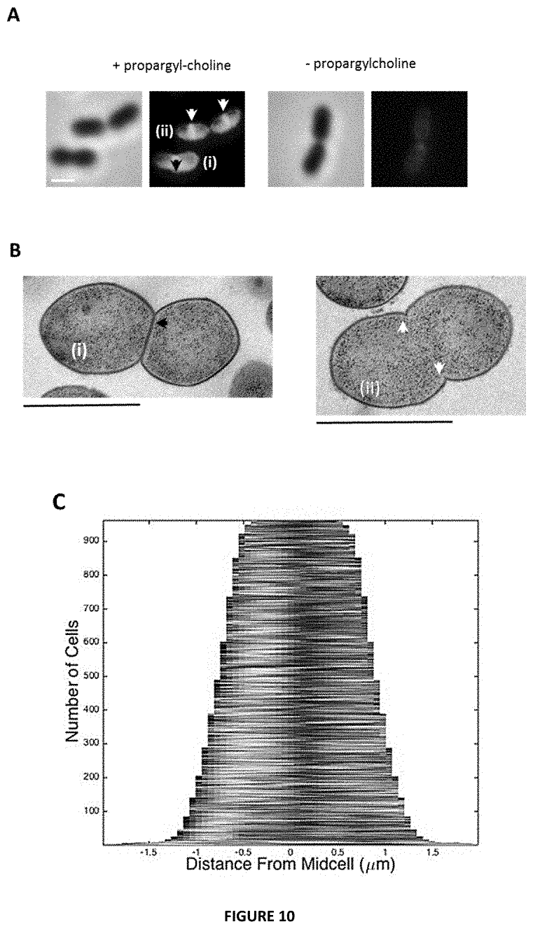

To obtain information on the TA biosynthesis dynamics, labeling of live pneumococcal cells have been performed after optimizing the reactional conditions. Reduction of copper concentration to 50 μM, addition of the catalyst THPTA and reducting the incubation time to 30 min allowed to specifically label TA (FIG. 10A). The fluorescence intensity displayed by pneumococcal cells grown in presence of propargyl-choline was measured and compared to the signal detected on cells grown with choline in five independent experiments (n=155 to 2445 in each experiment and for each culture condition). The fluorescent signal ratio propargyl-choline/choline was 4.28±0.8.

Fluorescence detected at the cell periphery and at the contact zone between daughter cells before they separate suggest a membrane localization of TA/LTA (FIG. 10A, stage (i), black arrow head). An electron micrograph of a pneumococcal cell of similar morphology is shown to appreciate in details the membrane topology (FIG. 10B, stage (i), black arrow head). Early-division stage (ii) is characterized by the on-set of cross-wall synthesis and membrane invagination (FIGS. 10A and 10B, white arrow heads). TA labeling is observed at the septal site in these cells indicating that TA are synthesized and/or flipped across the membrane at the same time and the same place as peptidoglycan synthesis. Image analysis of a cell population confirms the septal localization of the TA labeling on pneumococcal cells over the cell cycle (FIG. 10C): after integration of the fluorescence signal in each cell, cells were sorted by their length value and the fluorescence values was plotted as a heat map.

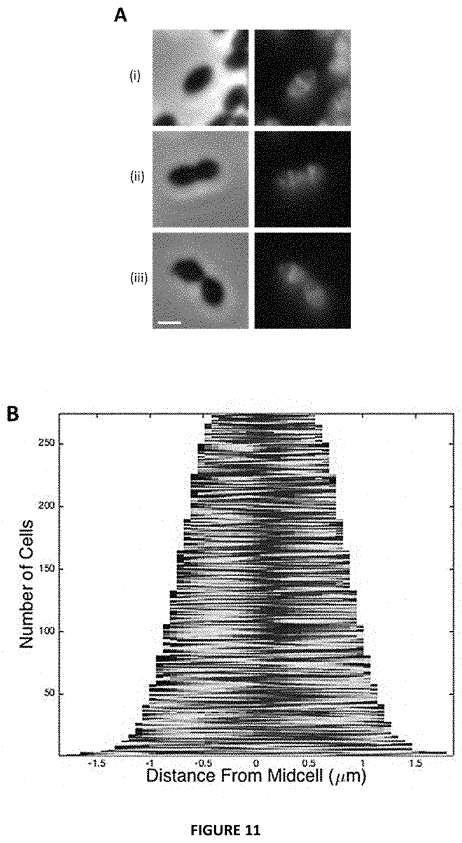

A pulse of propargyl-choline was performed. Pneumococcal cells were grown in medium containing choline, washed, incubated in presence of propargyl-choline for 30 min and submitted to the bioorthogonal reaction (FIG. 11). In these conditions, only TA synthesized during the 30 min pulse are labeled. Reduced membrane labeling (when compared to the 3 h culture period shown in FIG. 8) together with the septal site localization (FIGS. 11A and 11B) confirm that TA synthesis takes place in a relatively short time scale.

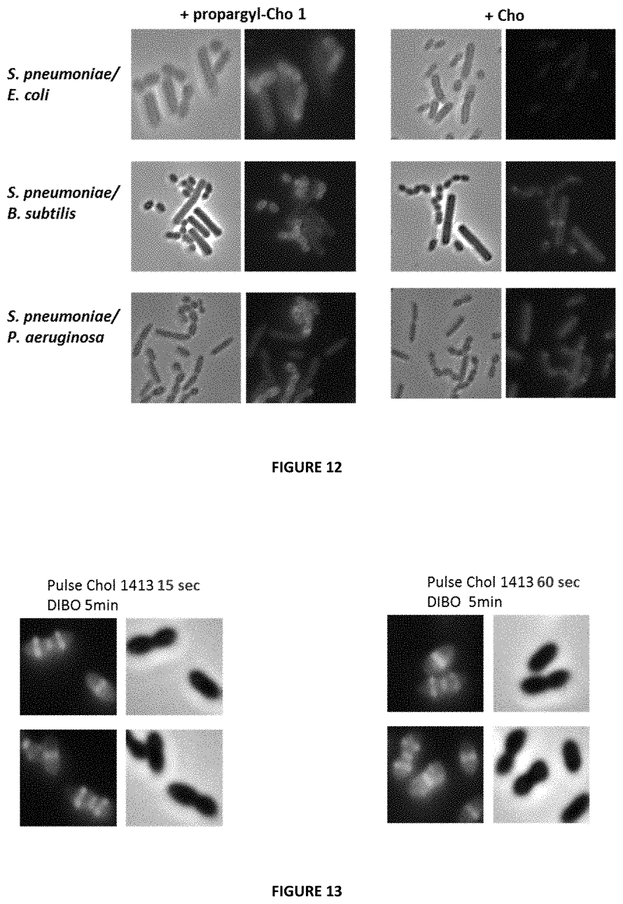

No fluorescent signal was detected when the bioorthogonal reaction was performed on Escherichia coli, Bacillus subtilis and Pseudomonas aeruginosa species, respectively Gram-negative and Gram-positive bacteria that do not metabolize choline (FIG. 12). This work is the first report of metabolic labeling of Gram-positive TA. These results demonstrate the specificity and selectivity of the labeling based on click chemistry. Long and short labeling pulses with propargyl-choline label TA in live cells at the septal site show that TA synthesis might be correlated to the peptidoglycan synthesis and the cell division. The success of this method offers possibility to explore mechanistic issues of pneumococcal TA biosynthesis in a more physiological context.

### Example 5. High-Speed Labeling of S. pneumoniae

A quick incubation of 15 seconds with the 1-azidoethyl-choline, followed by the addition of DIBO-ATTOS 488 for 5 min, is enough to obtain a high-quality labeling (FIG. 13).

### Example 6. “One-Pot” Two-Step Metabolic Labeling of S. pneumoniae

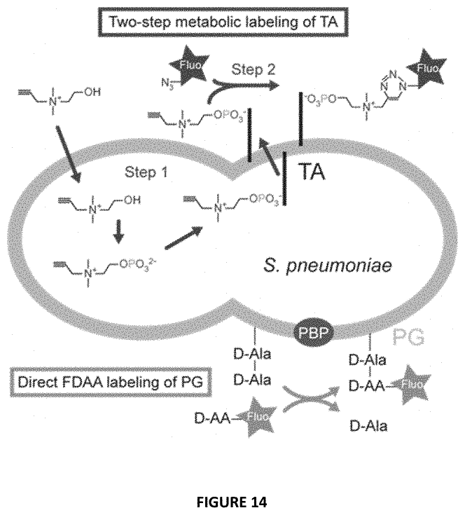

To monitor PG assembly, a recent method relies on the ability of bacteria to incorporate fluorescent D-amino acids (FDAA) into the growing PG (FIG. 14, direct labeling of PG). This labeling is thought to be catalyzed by the penicillin-binding proteins (PBPs) that normally catalyze the PG cross-linking. FDAAs are incorporated in a few minutes allowing labeling pulses that are significantly shorter than the bacterial generation time.

A comparable fast labeling method of TA was lacking. As shown in Example 4, the incorporation of choline-derivatives in TA of S. pneumoniae can be used to metabolically label TA in this organism (FIG. 14, two-step labeling of TA). However, this two-step approach is suited to uniformly label TA at the cell surface but is not optimal to pulse-chase experiments, as the Cu-catalyzed click reaction may be too slow relatively to the bacterial growth rate.

To study the relationship between the insertion of PG and TA in the cell wall, it is necessary to visualize both polymers with markers that can be incorporated at similar rates. Thus, to circumvent the lack of direct labeling method for TA, the Inventors have developed a choline-based two-step metabolic labeling of TA that allows pulse-chase experiments comparable to the direct method of PG labeling. This approach combines the rapid kinetics of a strain promoted azide alkyne cycloaddition (SPAAC) with the speed of a biological process.

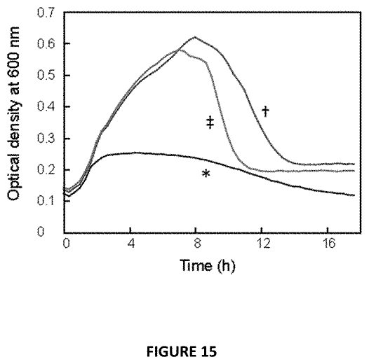

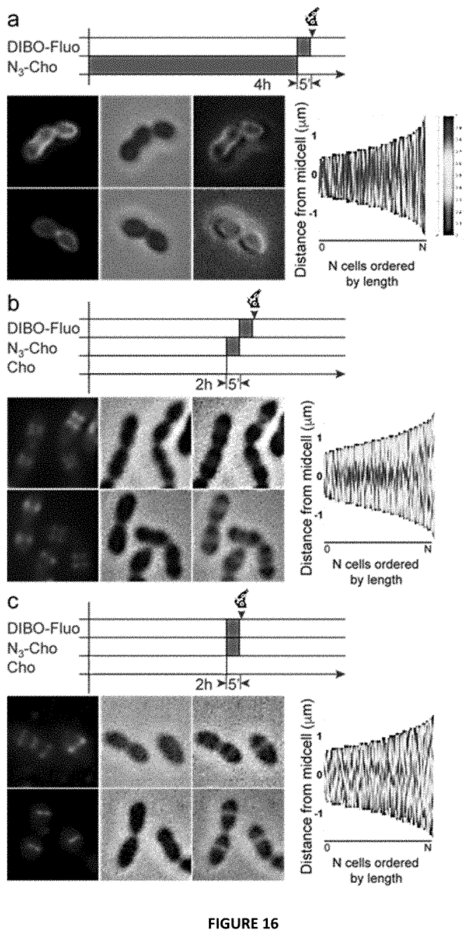

The choice was oriented towards a rapid click reaction involving an azide function carried by the choline and the strained dibenzoannulated cyclooctyne DIBO carrying the fluorophore. Azido-choline efficiently replaced choline to allow growth of pneumococcus (FIG. 15). For subsequent click reaction, fluorescent DIBO offers a compromise between stability and rapidity with a choice of commercially available fluorophores. Uniform cell surface labeling was obtained when cells were grown in the presence of azido-choline for 4 hours prior to fast bio-orthogonal labeling with Alexa Fluor® DIBO in 5 min (FIG. 16a).

Pulse-chase experiments have been performed using the two-steps approach. After growing cells in rich medium, washing cells to remove choline, the labeling pulse was carried out by adding successively the azido-choline alone and 5 min later the DIBO probe. The chase was initiated by dilution in rich medium. Cells could be adequately imaged at various chase times, but the method was not appropriate to document labeling at the pulse time (t=0). As cells continue to grow and build cell wall during the first step of the labeling process, adding the DIBO reagent even immediately after the azido-choline pulse, produced chase-like patterns with segregated bands of labeling (FIG. 16b).

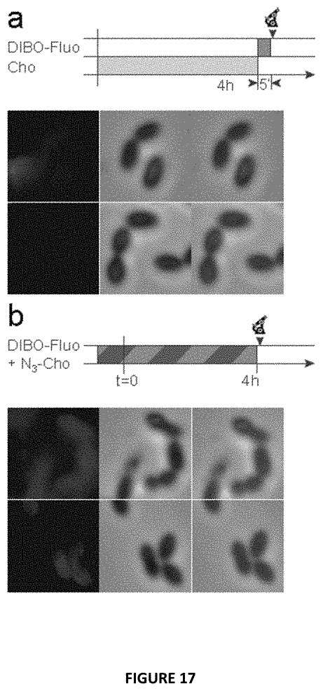

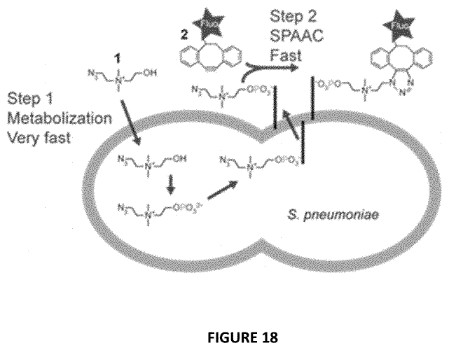

Then, it was attempted to add both the metabolic and fluorescent labeling agents at the same time. This “one-pot” approach allowed proper recording of cell wall expansion and TA insertion during the 5 min pulse time (FIG. 16c). No labeling and negligible background fluorescence were obtained with unmodified choline (FIG. 17). Pre-incubation together of the “clickable” reagents also abolished labeling, likely because the resulting fluorescent choline cannot be imported and/or metabolized (FIG. 17). Therefore, when both azido and DIBO reagents were added together during the labeling pulse, the observed signal must result from the rapid metabolization and presentation of azido-choline at the cell surface followed by its reaction with the DIBO-fluorophores (FIG. 18). At the reagents concentrations used, the “click” reaction in solution was slow enough to allow import of unreacted free azido-choline by the bacteria, while fast enough to modify TA-exposed azido-choline during the pulse time.

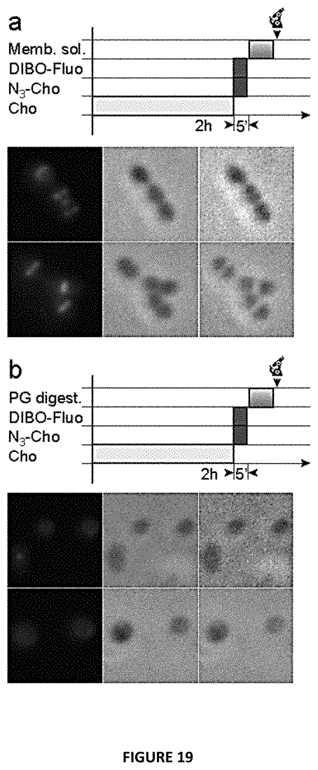

When “one-pot” pulse-labeled cells were treated either to remove the membrane or the cell wall, site-defined labeling of the sacculi was maintained, whereas labeling of the spheroplasts membrane was uniform, respectively (FIG. 19). As expected WTA attached to the PG are not mobile, while LTAs can diffuse laterally in the membrane, at least in the absence of cell wall.

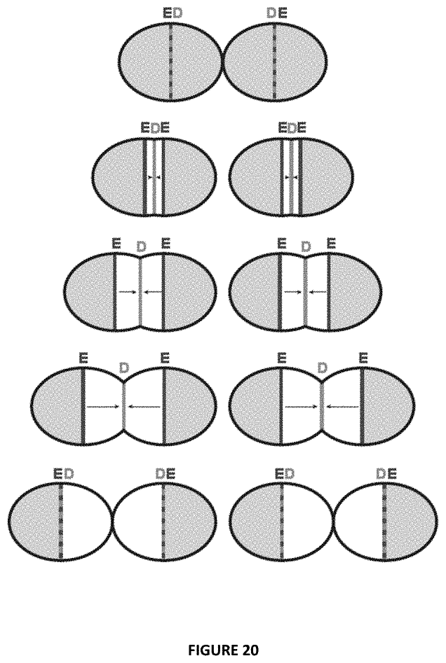

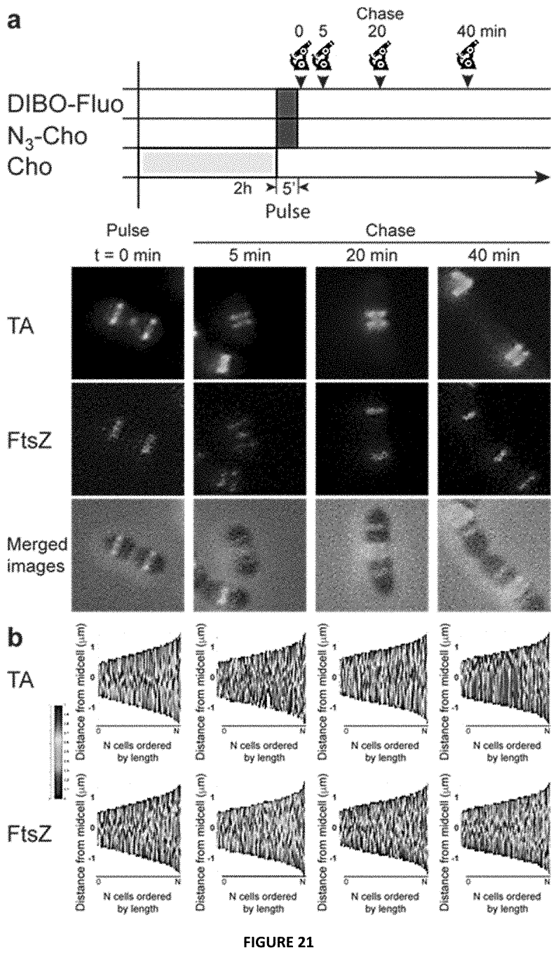

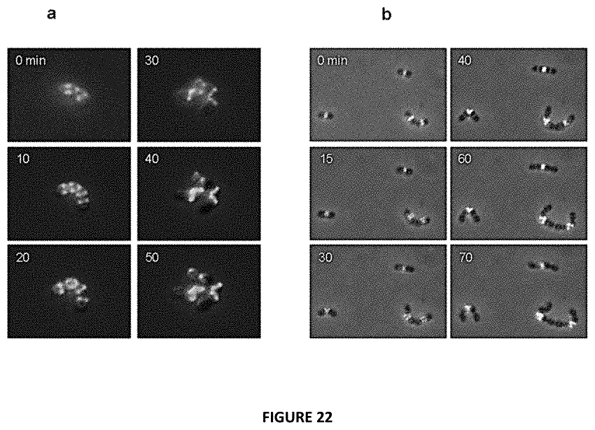

The “one-pot” two-step method was then applied in pulse-chase experiments to determine the localization and timing of TA insertion with respect to the cell cycle. A scheme of the cell cycle with a nomenclature of the main structural features is given in FIG. 20. A pulse-chase experiment was performed in a strain expressing a fluorescent FtsZ to serve as a marker of the cell division. During the 5 min-pulse, the new TA were mostly co-localized at the division site with the FtsZ-ring (FIG. 21a, t=0). However, in cells in late stages of division, FtsZ was not detected at the old division site, while new TA were still incorporated there. Indeed, the demographs show that FtsZ is re-localized to the equators of the daughter cells earlier than is the insertion of new TA (FIG. 21b, t=0). The localization of FtsZ was that at the time of the observation, whereas the localization of the labeled TA was that of TA incorporated during the 5 min-pulse. Although cells were imaged right after labeling and washing, it cannot be excluded that the difference observed between FtsZ and the new TA is due to this short delay. When imaged after a chase of 5 min, the TA labeling was observed as having spread or split and segregated on each side of the division site (FIG. 21a, t=5 min). The latter pattern is exemplified by the presence of FtsZ, at the time of imaging between parted bands of TA labeled during the pulse. Once the cell division is complete, as observed after 20 and 40 min (FIG. 21, t=20 and 40 min), the segregated bands of labeled TA remain at the same distance of each other if cells are forming chains. If cells have separated after their division, the TA labeling is found on free hemispheres, as shown in the demographs on one side of many cells. This behavior was also observed by time-lapse microscopy (FIG. 22).

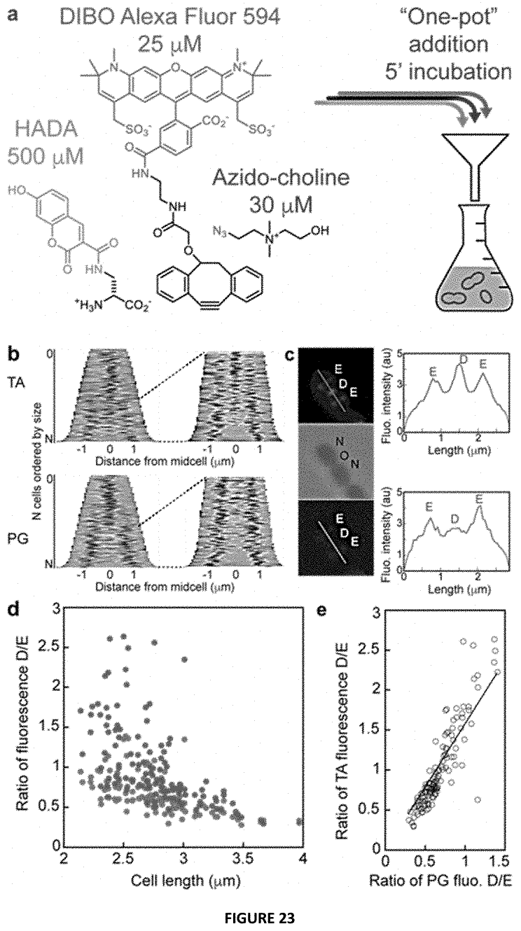

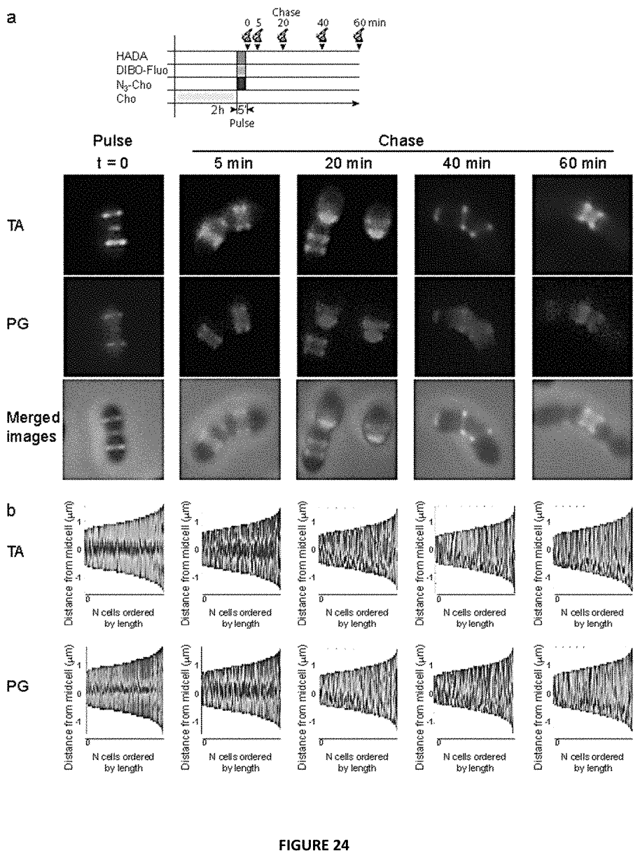

The “one-pot” approach was the applied in conjunction with direct labeling with FDAA to directly compare TA insertion and PG synthesis. Dual pulse-chase labeling experiments were performed to compare the localization and timing of both processes (FIG. 23a; FIG. 24). Overall, surface presentation of new TA that were bio-orthogonally labeled was concomitant with the activity of PBPs responsible for the PG labeling. The “one-pot” metabolic method for labeling TA is therefore as fast as the direct method of PG labeling with FDAA. However, a difference between the two labeling is noticeable immediately after of the pulse time (t=0): simultaneous TA-labeling at closing division sites (D) or pole tips and at equators (E) is observed more often than simultaneous PG-labeling at those two sites (FIG. 23b). To quantify this observation, the fluorescence data for cells (di-cocci) that were longer than 2.1 μm was analyzed by measuring the ratio of signal at the old division site (D) over that at the equators (E, future division sites). When this ratio was plotted against the cell length, it appeared that the fluorescence at the old division site due to TA-labeling was more important than that due to PG labeling. This difference diminished as cells became longer and approached cell separation (FIGS. 23c and 23d). When the D/E PG-labeling ratio was plotted against the D/E TA-labeling ratio, the data could be fitted to a straight line of slope 2.0±0.1 (standard error) that was significantly different from the value of 1 expected without difference between the distribution of both labeling (FIG. 23e), indicating that insertion of TA persists at the division site after PG synthesis has ceased.

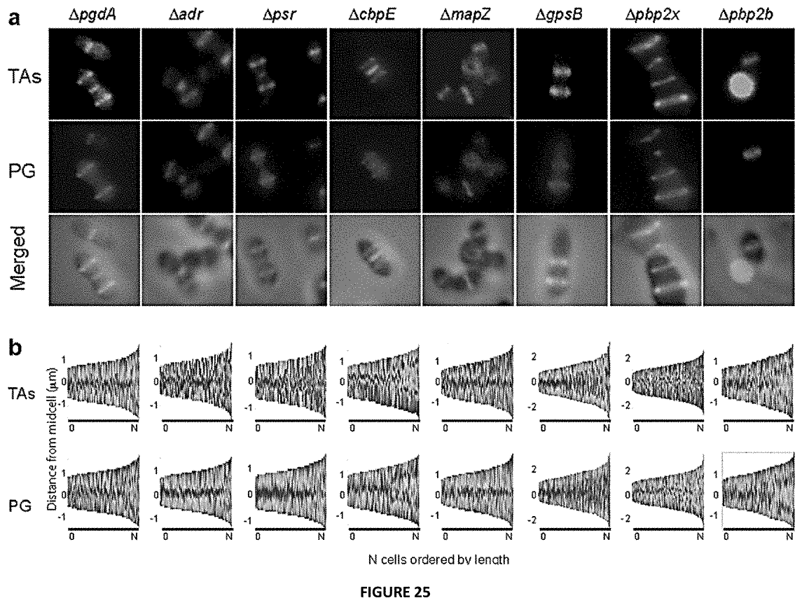

Numerous mutations are known to affect the cell wall or the morphology. A simultaneous pulse-labeling of TA and PG in various mutant strains was performed to determine whether the tight coordination of these processes was affected (FIG. 25). The reasons underlying the choice of mutations are discussed in the supplementary material. The individual deletion of pgdA, adr, psr, cbpE, mapZ, gpsB, or the depletion of pbp2x and pbp2b, did not affect the co-localization of TA insertion and PG assembly.

Many bacteria in laboratory cultures have generation times shorter than an hour. To study the dynamics of molecular events at the cell surface with pulse-chase experiments therefore requires short labeling pulses. It has been found here that if metabolization of the azido-compound (first step) is significantly faster than the SPAAC reaction (second step), both reagents can be added simultaneously, making the method comparable to direct metabolic approaches.

The “one-pot” method is compatible with other labeling techniques and co-labeling confirmed that TA insertion and PG assembly are largely overlapping, but careful quantification revealed that incorporation of TA persists at the division site later than PG assembly.

Further insights in bacterial cell wall biology will arise by improving the spatial resolution of imaging, which is certainly possible since our approach can be used in principle with SPAAC-linked fluorophores compatible with direct stochastical optical reconstruction microscopy (dSTORM). The size of the functions tolerated on modified nutriment is generally small and is therefore a limiting factor of direct metabolic labeling methods, while dSTORM fluorophores are generally large. The new “one-pot” method provides a simple alternative to the problem of label size applied to the study of rapid metabolic pathways in bacteria and other organisms.

1. A method of labeling a bacterium that is able to metabolize choline, said method comprising a step (i) of incubating the bacterium in a culture medium containing a modified choline which is metabolized by the bacterium and covalently associated to a teichoic acid (TA) in the cytoplasm before being exported and integrated into the cell wall of the bacterium.

2. The method according to claim 1, further comprising a step (ii) of contacting the bacterium with a tag molecule to generate a binding reaction between the modified choline bound to the teichoic acid (TA) present in the cell wall of the bacterium and the tag molecule.

3. The method according to claim 2, wherein the modified choline comprises at least one reactive group X allowing the binding of the modified choline to the tag molecule, said at least one reactive group X being selected from the reactive groups consisting of an alkene group, an alkyne group, an azide group, a cyclopropenyl group and a diazirine group.

4. The method according to claim 2, wherein the modified choline is selected from the group consisting of:
Propargyl-choline,
1-azidoethyl-choline,
Allyl-choline,
cyclopropyl-choline,
azidoethyl-diazinebutyl-choline.

5. The method according to claim 2, wherein the tag molecule is selected from the group consisting of a fluorescent molecule, a luminescent molecule, a radioactive molecule, a biotin molecule or a derivative thereof and an antigen molecule.

6. The method according to claim 2, wherein the tag molecule comprises at least one reactive group Y allowing its binding to the modified choline, said at least one reactive group Y being preferably selected from the group consisting of an alkene group, an alkyne group, an azide group, a cyclopropenyl group, a tetrazine group, a dibenzocyclooctyl (DBCO) group, a dibenzocyclooctine (DIBO) group, a bicyclononine (BCN) group, a Trans-Cyclooctene (TCO) group, a strained Trans-Cyclooctene (sTCO) group.

7. The method according to claim 2, wherein the tag molecule is selected from the group consisting of:
3-Azido-7-(diethylamino)-2H-chromen-2-one, and
7-nitro-N-(prop-2-yn-1-yl)benzo[c][1,2,5]oxadiazol-4-amine.

8. The method according to claim 2, wherein said steps (i) and (ii) are performed simultaneously.

9. The method according to claim 2, further comprising a step (iii) of detection and/or quantification of the bacterium by detecting and/or quantifying the tag molecule bound to the bacterium.

10. The method according to claim 1, wherein the modified choline is chemically modified to incorporate a radioactive isotope.

11. The method according to claim 1, wherein said bacterium is a Gram-positive bacterium or a Gram-negative bacterium.

12. An in vitro method of tracking a bacterium by bio-imagery comprising labeling the bacterium with a tag molecule according to the method of claim 1, thereby allowing the tracking of the bacterium.

13. An in vitro method for the diagnosis of a bacterial infection from a biological sample of a patient comprising labeling the bacterium responsible for the infection with a tag molecule according to the method of claim 1, thereby allowing the detection of the bacterium.

14. A method for the preparation of a vaccine composition containing a bacterium or fragments thereof, comprising labeling the bacterium in vitro with an antigen according to the method of claim 2, said antigen being bound to the bacterium or to the fragments thereof.

15. An in vitro method for the preparation of a bio-material comprising:
a step of labeling a first population of bacteria with a first modified choline by using the labeling method of claim 2,
a step of labeling a second population of bacteria with a second modified choline by using the labeling method of claim 2,
the first modified choline used to label the first population and the second modified choline used to label the second population being respectively chosen to cross-react by Click chemistry between each other, followed by
a step of binding the first population with the second population by click chemistry to form a bio-material.

16. An in vitro method of identifying an agent that inhibits the bacterial cell wall synthesis, said method comprising:
a step of contacting a bacterium with a test agent,
a step of labeling the bacterium using the method of claim 2,
wherein a test agent that inhibits the labeling of the bacterium is considered a candidate agent for inhibiting the cell wall synthesis of the bacterium.

17. A kit to label a bacterium comprising:
a modified choline that (i) comprises at least one reactive group X allowing the binding of the modified choline to the tag molecule, said at least one reactive group X being selected from the reactive groups consisting of an alkene group, an alkyne group, an azide group, a cyclopropenyl group and a diazirine group, or (2) is selected from the group consisting of Propargyl-choline, 1-azidoethyl-choline, Allyl-choline, cyclopropyl-choline, and azidoethyl-diazinebutyl-choline,
a tag molecule that (i) is selected from the group consisting of a fluorescent molecule, a luminescent molecule, a radioactive molecule, a biotin molecule or a derivative thereof and an antigen molecule, (ii) comprises at least one reactive group Y allowing its binding to the modified choline, said at least one reactive group Y being preferably selected from the group consisting of an alkene group, an alkyne group, an azide group, a cyclopropenyl group, a tetrazine group, a dibenzocyclooctyl (DBCO) group, a dibenzocyclooctine (DIBO) group, a bicyclononine (BCN) group, a Trans-Cyclooctene (TCO) group, a strained Trans-Cyclooctene (sTCO) group, or (iii) is selected from the group consisting of 3-Azido-7-(diethylamino)-2H-chromen-2-one, and 7-nitro-N-(prop-2-yn-1-yl)benzo[c][1,2,5]oxadiazol-4-amine, and
a culture medium allowing the growth of the bacterium.

18. The method according to claim 2, wherein said binding reaction is a Click chemistry reaction.

19. The method according to claim 11, wherein the bacterium is a Gram-positive bacterium selected from the Streptococcus genus, or wherein the bacterium is a Gram-negative bacterium selected from Haemophilus or Neisseria genera.

20. The method of according to claim 19, wherein the bacterium is a Gram-negative bacterium selected from S. pneumoniae, H. influenzae and Neisseria ssp.
